(19) United States
(12) Patent Application Publication
(10) Pub. No.: US 2026/0083133 A1
(43) Pub. Date: Mar. 26, 2026

(54) Application of Iron-phosphorus Nanomaterial in Promoting Preservation of Pepper

(71) Applicant: Jiangnan University, Wuxi (CN)

(72) Inventors: Le YUE, Wuxi (CN); Zhenyu Wang, Wuxi (CN); Chuanxi Wang, Wuxi (CN)

(21) Appl. No.: 19/398,334

(22) Filed: Nov. 24, 2025

Related U.S. Application Data
(63) Continuation of application No. PCT/CN2025/094147, filed on May 12, 2025.

(30) Foreign Application Priority Data
May 15, 2024 (CN) 2024106017279

(51) Int. Cl. A01N 59/26; A01P 1/00; A01P 21/00; B82Y 5/00

(52) U.S. Cl. CPC A01N 59/26; A01P 1/00; A01P 21/00; B82Y 5/00

(57) ABSTRACT

Disclosed is application of an iron-phosphorus nanomaterial in promoting the preservation of pepper, belonging to the field of a novel pesticide. According to the application of an iron-phosphorus nanomaterial in promoting the preservation of pepper of the disclosure, the iron-phosphorus nanomaterial is made into an iron-phosphorus nanomaterial solution, which is applied onto leaves of pepper plants through foliage spray in a flowering period of pepper. The iron-phosphorus nanomaterial has a width of 60 nm to 100 nm, a hydraulic diameter of 196.97±55.43 nm, and a Zeta potential of 16.33±0.80 mV. A concentration of the iron-phosphorus nanomaterial solution is 1 mg/L to 50 mg/L. The disclosure promotes the preservation of pepper through the foliage application of the iron-phosphorus nanomaterial solution.

### TECHNICAL FIELD

The disclosure relates application of an iron-phosphorus nanomaterial in promoting preservation of peppers, and belongs to the field of a novel pesticide.

### BACKGROUND

Globally, about 1.3 billion tons of foods are wasted each year due to biotic factors (insects, pests, rodents, fungi and bacteria) and abiotic factors (temperature, humidity and rain). Among these, spoilage accounts for a striking 33% of fruits and vegetables. Effectively reducing post-harvest losses of fruits and vegetable plays an important role in sustainably feeding the global population. Peppers are one of the important vegetable crops worldwide, rich in various nutrients such as carotenoids, protein and vitamin C. However, peppers are prone to spoilage at various stages from field harvesting to post-harvest commercial handling. Therefore, reducing post-harvest losses is crucial for sustainably feeding the global population.

Conventional preservation techniques for fruits and vegetables mainly include physical methods (such as modified atmosphere packaging, low-temperature refrigeration, irradiation, heat treatment, ultrasound, high-voltage electrostatic fields), chemical methods (achieving a preservation by spraying or soaking the fruits or vegetables with some chemical reagents, and commonly used chemical reagents mainly include methyl jasmonate and 1-methylcyclopropene) and biological methods (performing post-harvest preservation treatment on vegetables and fruits using substances extracted from different animals, plants and microorganisms or substances obtained through other means). However, conventional preservation techniques for vegetables and fruits have defects of high cost, complicated operation, low safety, etc. Therefore, there is an urgent need to develop a novel sustainable and environmental-friendly preservation technique to meet consumers' emphasis on food safety and their demands on food freshness.

### SUMMARY

### Technical Problem

The technical problem is to provide a novel pepper preservation method with the advantages of sustainability, environmental-friendly effect, simple operation and low cost.

### Technical Solution

In order to solve the above problem, the disclosure makes an iron-phosphorus nanomaterial into a solution used as a preservation material to be applied onto leaves of pepper plants through foliage spray for culture, so as to promote the preservation of pepper fruits.

The first objective of the disclosure is to provide a method for promoting preservation of pepper using an iron-phosphorus nanomaterial. According to the method, the iron-phosphorus nanomaterial is made into an iron-phosphorus nanomaterial solution to be applied onto leaves of pepper plants through foliage spray.

In an embodiment of the disclosure, the iron-phosphorus nanomaterial has a width of 60 nm to 100 nm, a hydraulic diameter of 196.97±55.43 nm, and a Zeta potential of 16.33±0.80 mV.

In an embodiment of the disclosure, the iron-phosphorus nanomaterial solution is an iron-phosphorus nanomaterial aqueous solution having a concentration of 1 mg/L to 50 mg/L, preferably 10 mg/L.

In an embodiment of the disclosure, an application amount of the iron-phosphorus nanomaterial is 5 mL/plant, an application frequency is once per day, totaling 4 times, spraying time is 9 a.m. to 11 a.m., and the uniform spray application onto the front leaf surfaces and back leaf surfaces is possibly ensured.

In an embodiment of the disclosure, the application period is a seedling period, a flowering period, or a fruiting period, preferably a flowering period.

In an embodiment of the disclosure, no rain within 4 h after the application of the iron-phosphorus nanomaterial solution is possibly ensured.

In an embodiment of the disclosure, a method for preparing the iron-phosphorus nanomaterial includes the following steps:
respectively dissolving polyvinylpyrrolidone, a water-soluble trivalent iron salt, and a water-soluble phosphorus source in water to obtain a polyvinylpyrrolidone solution, an iron salt solution, and a phosphorus source solution; and
mixing the polyvinylpyrrolidone solution with the phosphorus source solution, then, dropwise adding the iron salt solution while stirring, after dropwise addition, continuously stirring for reaction for a period of time, then performing centrifugation, collecting a precipitate, transferring the precipitate into a reaction vessel, adding water for taking a hydrothermal reaction, after the reaction is completed, performing centrifugation, collecting solids, and performing washing and drying to obtain the iron-phosphorus nanomaterial.

In an embodiment of the disclosure, in the method for preparing the iron-phosphorus nanomaterial, a mass ratio of the polyvinylpyrrolidone to the water-soluble trivalent iron salt is 1:(4-5), specifically and optionally 1:4.05.

In an embodiment of the disclosure, in the method for preparing the iron-phosphorus nanomaterial, a mass ratio of the water-soluble trivalent iron salt to the water-soluble phosphorus source is 1:(1.5-2.5), specifically and optionally 1:1.7.

In an embodiment of the disclosure, in the method for preparing the iron-phosphorus nanomaterial, a molar ratio of iron in the water-soluble trivalent iron salt to phosphorus in the water-soluble phosphorus source is 0.25:1.

In an embodiment of the disclosure, in the method for preparing the iron-phosphorus nanomaterial, a concentration of the polyvinylpyrrolidone solution is 0.05 g/mL.

In an embodiment of the disclosure, in the method for preparing the iron-phosphorus nanomaterial, a concentration of the phosphorus source solution is 0.1 g/mL.

In an embodiment of the disclosure, in the method for preparing the iron-phosphorus nanomaterial, a concentration of the iron salt solution is 0.15 g/mL to 0.25 g/mL.

In an embodiment of the disclosure, in the method for preparing the iron-phosphorus nanomaterial, a speed of dropwise adding the iron salt solution is one drop per 6 s.

In an embodiment of the disclosure, in the method for preparing the iron-phosphorus nanomaterial, the hydrothermal reaction is conducted at a temperature of 150° C. to 200° C. for a duration of 3 h to 8 h. Specifically and optionally, the hydrothermal reaction is conducted at 180° C. for 6 h.

In an embodiment of the disclosure, a method for preparing the iron-phosphorus nanomaterial specifically includes:
respectively weighing and dissolving 0.5 g of polyvinylpyrrolidone (PVP), 2.025 g of ferric chloride hexahydrate (Ill) (FeCl₃·6H₂O), and 3.45 g of ammonium dihydrogen phosphate (NH₄H₂PO₄) in 10 mL, 20 mL and 20 mL of deionized water;
mixing the obtained PVP solution and the NH₄H₂PO₄, placing the FeCl₃ solution in a separatory funnel, dropwise adding the FeCl₃ solution into the mixed solution at a speed of one drop per 6 s, while stirring with a magnetic stirrer in the whole process, after the dropwise addition, continuously stirring for 30 min, transferring the stirred suspension into a centrifuge tube to be subjected to centrifugation at 6000 rpm for 10 min, removing a supernatant, transferring a precipitate into a 100 mL stainless steel high-pressure reaction vessel with a polytetrafluoroethylene inner liner, and supplementing 60 mL of ultrapure water;
then the reaction is conducted at 180° C. for 6 h, after cooling, performing centrifugation at 5000 rpm for 5 min, collecting a precipitate, and washing the precipitate by deionized water for 3 times; and finally, performing drying in a vacuum drying oven at 60° C. for 2 h to obtain iron-phosphorus nanomaterial powder.

A second objective of the disclosure is to provide a method for improving contents of lignin, total phenols, flavonoids, and capsaicin in pepper fruits, and activities of antioxidant enzymes through promoting phenylpropane metabolism and capsaicin metabolism pathway changes of fruits. According to the method, an iron-phosphorus nanomaterial is made into an iron-phosphorus nanomaterial solution to be applied onto leaves of pepper plants through foliage spray.

The iron-phosphorus nanomaterial has a width of 60 nm to 100 nm, a hydraulic diameter of 196.97±55.43 nm, and a Zeta potential of 16.33±0.80 mV.

The iron-phosphorus nanomaterial solution is an iron-phosphorus nanomaterial aqueous solution having a concentration of 1 mg/L to 50 mg/L.

An application amount of the iron-phosphorus nanomaterial is 5 mL/plant, an application frequency is once per day, totaling 4 times, spraying time is 9 a.m. to 11 a.m., and the uniform spray application onto the front leaf surfaces and back leaf surfaces is possibly ensured.

An application period is a flowering period.

A third objective of the disclosure is to provide a method for promoting preservation of pepper based on decrease of relative abundances of microorganisms (Enterobacter and Chryseobacterium) related to decay in pepper fruits and increase of relative abundances of beneficial microorganisms (Pseudomonas, Arthrobacter, Sphingobacterium and Paenibacillus) in pepper fruits. According to the method, an iron-phosphorus nanomaterial is made into an iron-phosphorus nanomaterial solution to be applied onto leaves of pepper plants through foliage spray.

The iron-phosphorus nanomaterial has a width of 60 nm to 100 nm, a hydraulic diameter of 196.97±55.43 nm, and a Zeta potential of 16.33±0.80 mV.

The iron-phosphorus nanomaterial solution is an iron-phosphorus nanomaterial aqueous solution having a concentration of 1 mg/L to 50 mg/L.

An application amount of the iron-phosphorus nanomaterial is 5 mL/plant, an application frequency is once per day, totaling 4 times, spraying time is 9 a.m. to 11 a.m., and the uniform spray application onto the front leaf surfaces and back leaf surfaces is possibly ensured.

An application period is a flowering period.

### Beneficial Effects

(1) The disclosure improves the yield and quality of pepper through foliage application of the iron-phosphorus nanomaterial solution. After the treatment with 10 mg/L Fe—P NMs, the net photosynthetic rate (Pn), the transpiration rate (E), the intercellular carbon dioxide concentration (Ci), and the stomatal conductance (Gs) are respectively increased by 73.3%, 240.2%, 29.2% and 72.4%; and the chlorophyll content, the plant height, the shoot fresh weight, the root fresh weight, and the numbers of fruits are respectively increased by 21.8%, 13.0%, 36.1%, 31.0%, and 29.4%. Through the treatment with the 10 mg/L Fe—P NMs, the quality of the pepper fruits may be significantly improved, and the nutritive value and flavor may be kept during storage.
(2) Through the treatment with Fe—P NMs, the processes of phenylpropane metabolism, starch and sucrose metabolism, tricarboxylic acid (TCA) circulation, and amino acid metabolism of arginine and proline, cysteine and methionine, etc. in the pepper fruits are improved. In the phenylpropane pathway, after the treatment with 10 mg/L Fe—P NMs, the contents of caffeic acid, chlorogenic acid, protocatechuic acid, hesperetin, phloretin, and naringenin are increased by 34.3%, 97.2%, 65.6%, 36.8%, 22.7% and 288.6%, so that the synthesis of the capsaicin (improved by 65.6%) is further promoted. After the treatment with 10 mg/L Fe—P NMs, the activities of the antioxidant enzymes (POD, SOD, and CAT, respectively improved by 77.6%, 17.4% and 77.1%) in the pepper fruits are significantly improved, and the content of malondialdehyde (MDA) is reduced by 19.3%, so that the pepper fruits are protected from oxidative stress injury in the storage period.
(3) Through the treatment with the Fe—P NMs, the microflora structures of the pepper fruits are changed. At the genus level, relative abundances of microorganisms (Enterobacter and Chryseobacterium) related to decay are decreased, and relative abundances of beneficial microorganisms (Pseudomonas, Arthrobacter, Sphingobacterium and Paenibacillus) are increased. These microorganisms may effectively prevent and control the fruit diseases to help finally establish a micro-ecological environment favorable for preservation of pepper fruits.

### BRIEF DESCRIPTION OF FIGURES

FIG. 1A shows a TEM image of an iron-phosphorus nanomaterial (Fe—P NMs) in Example 1, and an FIG. 1B shows a XRD diagram of an iron-phosphorus nanomaterial (Fe—P NMs) in Example 1.

FIG. 2 shows pepper growth indexes under treatment with Fe—P NMs of different concentrations in different periods, the pepper growth indexes include the height of pepper plants, the number of pepper fruits, the net photosynthetic rate (Pn), the transpiration rate (E), the intercellular carbon dioxide concentration (Ci), and the stomatal conductance (Gs). (Note: ST, FT and MT respectively represent spray application in a seedling period, spray application in a flowering period, and foliage spray in a fruiting period). (Note: different letters in histograms show significant differences between groups (p<0.05); and indexes in one group of histograms in A to F are sequentially CK, 1 mg/L, 10 mg/L, and 50 mg/L from the left to the right).

FIG. 3 shows properties of pepper under different treatment, the properties of pepper include the net photosynthetic rate (Pn), the transpiration rate (E), the intercellular carbon dioxide concentration (Ci), the stomatal conductance (Gs), the chlorophyll content, the plant height, the shoot and root fresh weight, and the number of fruits. (Note: different letters in histograms show significant differences between groups (p<0.05); and indexes in one group of histograms in G are sequentially shoot and root from the left to the right).

FIG. 4 shows properties of pepper fruits under different treatment in a storage period, the properties of pepper fruits include the change of the chlorophyll content, the change of the soluble sugar content, the change of the vitamin C content, the change of the total phenolic content, and the change of the lignin content. (Note: different letters in histograms show significant differences between groups (p<0.05); and indexes in one group of histograms in A to F are sequentially CK, NMs, and Ion from the left to the right).

FIG. 5 shows the change of a metabolic profiling of pepper fruits after the treatment with Fe—P NMs. Different letters show significant differences between groups (p<0.05).

FIG. 6A shows relative abundances of predominant bacterial communities at the phylum level of the pepper fruits after the treatment with Fe—P NMs, and FIG. 6B shows relative abundances of main genera of predominant bacterial communities of the pepper fruits after the treatment with Fe—P NMs. Different letters show significant differences between groups (p<0.05).

### DETAILED DESCRIPTION

Exemplary examples of the disclosure will be illustrated hereafter. It should be understood that the examples are intended to provide a better explanation to the disclosure but are not intended to limit the disclosure.

### Test Method:

### 1. TEM Test:

A transmission electron microscope (TEM, JEM-2100, Nippon electronics Co, Japan) was used for observing the sizes and appearances of NMs.

### 2. Test on Hydrodynamic Diameter and Zeta Potential Analysis:

The test was performed using a Malvern nanoparticle size analyzer (Nano-ZS90, Malvern Instruments, UK) with ultrapure water as the fluid.

### 3. XRD Test:

X-ray diffraction (XRD, Germany Brock AXS Co. LTD) was adopted for analyzing Fe—P NMs chemical formula composition.

### 4. Growth Parameter Determination:

In the pepper plant growth period, when the second leaf of the pepper completely unfolded, photosynthetic parameters, such as the intercellular carbon dioxide concentration (Ci), the transpiration rate (E), the stomatal conductance (Gs), and the net photosynthetic rate (Pn) were measured in situ through a portable gas photosynthesis system (CIRAS-3, PP-Systems, USA). The determination time was 8 a.m. to 11 a.m. to eliminate the strong light effect. Besides, the plant heights and the chlorophyll contents (SPAD-502 plus, Konica Minolta Inc, Japan) were measured in situ. During sampling, shoot parts and root parts were separated, while fruits were picked, the shoot parts and the root parts were flushed by deionized water and were then dried through wiping, and the fresh weight (FW) was measured using a one ten-thousandth balance (OHAUS, Shanghai), and the number of fruits was recorded. The pepper plants were placed in a drying oven, were subjected to withering at 105° C. for 10 min, and were dried at 60° C. for 72 h to constant weight, and then, the dry weight (DW) of the sample was measured.

### 5. Nutritional Quality Determination on Fruits:

The total phenol substances in pepper fruits were extracted and determined using a Folin-Ciocalteu colorimetric method. In short, 20 mg of frozen pepper fruits were ground in liquid nitrogen, were mixed with 95% methyl alcohol, and were extracted for 48 h in darkness. The mixture was centrifugated at 25° C. and 12000 g for 5 min. A supernatant was sufficiently mixed with 200 μL of a 10% (v/v) Folin-Ciocalteu reagent. Na₂CO₃ was added, incubation was performed at a room temperature for 2 h, 200 μL of the sample was transferred onto a transparent 96-pore microwell plate, and the absorbance was measured at 765 nm. Standard curve drawing: a gallic acid (0.050 g) was dissolved by 100 mL of 95% methyl alcohol to obtain a gallic acid solution (500 μg mL⁻¹), and 0.1 mL, 0.3 mL, 0.5 mL, 1.0 mL, 2.0 mL, and 5.0 mL of the gallic acid solutions were respectively taken and added into volumetric flasks to reach a constant volume (50 mL) with 95% methyl alcohol.

Lignin extraction and determination method: 100 mg of fresh pepper fruits were ground into powder in liquid nitrogen, the powder was transferred into a 2 mL centrifuge tube, 1.5 mL of 95% ethyl alcohol was added, after centrifugation, a supernatant was removed, the sample was washed, and the operation was repeated for three times. After the centrifugation, a precipitate was collected. After drying in the air, 0.2 mL of a 25% acetyl bromide (acetyl bromide:glacial acetic acid=25:75, v/v) solution was added, the material was placed in a 70° C. water bath for reaction for 30 min, and inverted mixing was performed once every 10 min to ensure the sufficient immersion of the precipitate in the solvent. 0.16 mL of 2 mol L⁻¹ NaOH was added for terminating the reaction. Then, 2 mL of glacial acetic acid and 0.04 mL of hydroxylamine hydrochloride (521.175 g L⁻¹) were added into the sample, uniform shaking was performed, and 1000 rpm centrifugation was performed for 10 min. Finally, 0.1 mL of a supernatant was taken, and 2.0 mL of glacial acetic acid was added for dilution. The absorbance of the mixture was measured at 280 nm. Standard curve manufacturing: lignin was used for preparing lignin solutions with the concentrations of 0, 1, 2.5, 5, and 10 mg L⁻¹, then, the absorbance was measured at 280 nm. The absorbance of the measured sample was substituted into a standard curve formula to calculate the lignin contents of different treatment groups.

The soluble sugar was determined using an anthrone colorimetric method.

The vitamin C was determined using a 2,6-dichlorophenol indophenol titration method.

A method for determining the chlorophyll content: about 100 mg of fresh pepper fruit tissues were weighed, and were placed in a 2 mL centrifuge tube, 1 mL of an 80% acetone solution was added for sufficient vortex, and centrifugation was performed at 4° C. and 12000 rpm for 20 min. Finally, a supernatant was collected, and the absorbance was respectively measured at wavelengths of 645 nm and 663 nm. The chlorophyll content in the pepper fruits was calculated using the following formula: the chlorophyll content (g kg⁻¹)=[(20.29A₆₄₅+8.05A₆₆₃)×V]/M. In the formula, A₆₄₅ and A₆₆₃ were respectively the absorbances measured at wavelengths of 645 nm and 663 nm, V is a total volume (mL) of an extraction solution; and M is a mass (g) of the sample.

### 6. Test on Metabolite:

The harvested pepper fruit fresh samples were uniformly ground in liquid nitrogen. 100 mg of the material was taken and placed in a 2 mL centrifuge tube. 1.5 mL of 80% methyl alcohol aqueous solution was added. The methyl alcohol aqueous solution contained 0.1% formic acid and 0.2 mg L⁻¹ of 2-chlorophenylalanine as interior labels. The mixed solutions were uniformly mixed, ice bath ultrasonic treatment was performed for 30 min (35 kHz), centrifugation was performed for 15 min (4° C., 12000 rpm), a supernatant was subjected to vacuum spin-drying using a rotary evaporation concentrator (4° C.), then, 200 μL of methanol and acetonitrile water (4:4:2) was used for dissolution again, and centrifugation was performed for 10 min (4° C., 12000 rpm). The above supernatant (150 μL) was taken, and a metabolite was analyzed and determined through high performance liquid chromatography-mass spectrometry (HPLC-MS/MS, Thermo Scientific, Germany). All samples of the same volumes were mixed to obtain a quality control sample (QC), and a blank control was an 80% methyl alcohol solution. A mobile phase was (A): a 0.1% formic acid aqueous solution and (B): a 0.1% formic acid in acetonitrile. A chromatographic column was in a type of UPLC HSS T3 (1.7×100 mm, 1.8 μm, Waters), and a column temperature was 35° C. A flow velocity of the mobile phase was 0.35 mL min⁻¹, and a sample amount was 5 μL. A gradient elution mode was 0 min: 5% B; 1.5 min: 5% B; 10 min: 100% B; 11 min: 100% B; 11.5 min: 5% B; 14 min: 5% B.

### 7. Determination on Fruit Microorganisms

On the 21^{st} day during post-harvest storage of the pepper fruits, the fruits were placed in liquid nitrogen to be stored, and were then delivered to Wekemo Tech Group Co., LTD (Shenzhen, China) for sequencing analysis. Firstly, DNA extraction and detection were performed, then, V5-V7 regions of bacteria 16S rRNA were subjected to PCR amplification through a forward primer 799F (AACMGGATTAGATACCCKG) and a reverse primer 1193R (ACGTCATCCCCACCTTCC). After product purification, library preparation and library searching, sequencing was performed in an Illumina platform.

### Example 1

A method for preparing Fe—P NMs, including the following steps:
0.5 g of polyvinylpyrrolidone (PVP), 2.025 g of ferric chloride hexahydrate (III) (FeCl₃·6H₂O), and 3.45 g of ammonium dihydrogen phosphate (NH₄H₂PO₄) were weighed and respectively dissolved in 10 mL, 20 mL and 20 mL of deionized water;

the obtained PVP solution and the NH₄H₂PO₄ solution were mixed into a 250 mL round-bottom flask, the FeCl₃ solution was placed in a separatory funnel, and was dropwise added into the mixed solution at a speed of one drop per 6 s, while stirring was performed with a magnetic stirrer in the whole process, after the dropwise addition, stirring was continuously performed for 30 min, the stirred suspension was transferred into a centrifuge tube to be subjected to centrifugation at 6000 rpm for 10 min, a supernatant was removed, a precipitate was transferred into a 100 mL stainless steel high-pressure reaction vessel with a polytetrafluoroethylene inner liner, 60 mL of ultrapure water was supplemented, the reaction vessel was placed at 180° C. and maintained for 6 h, after cooling, centrifugation was performed at 5000 rpm for 5 min, a precipitate was collected, and was washed by deionized water for 3 times; and finally, drying was performed in a vacuum drying oven at 60° C. for 2 h to obtain iron-phosphorus nanomaterial powder recorded as Fe—P NMs.

The obtained Fe—P NMs was subjected to property test, and the test results were as follows:

TABLE 1
Hydraulic diameter and a Zeta potential
of nano manganese ferrate (MnFe₂O₄ NMs)

| Parameter | Fe—P NMs |
|---|---|
| Hydraulic diameter (nm) | 196.97 ± 55.43 |
| Zeta potential (mV) | 16.33 ± 0.80 |

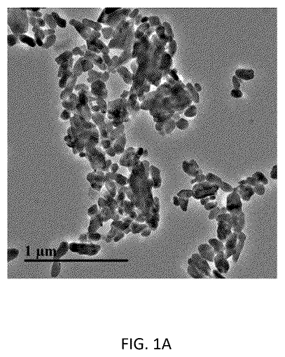

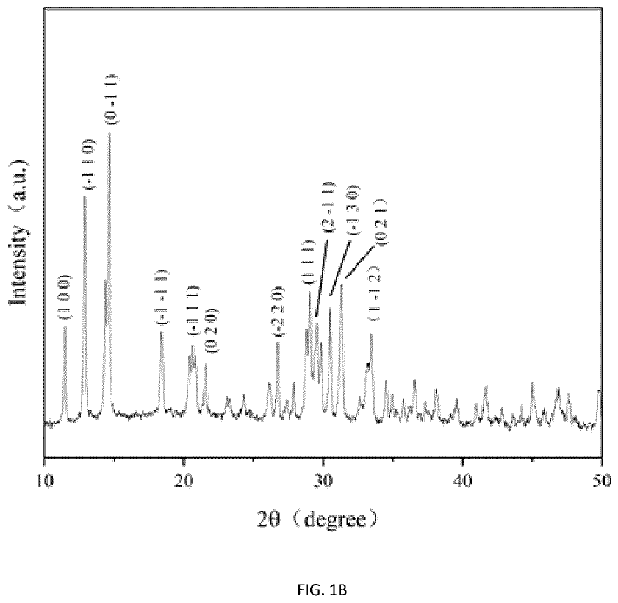

From FIG. 1A-FIG. 1B, it could be seen that the Fe—P NMs showed a rodlike structure with a width about 60 nm. The material was subjected to phase analysis using XRD, and the characteristic peaks matched well with those on the standard reference card for Fe₇(PO₄)₆.

### Example 2

A method for promoting preservation of pepper fruits using Fe—P NMs prepared in Example 1, including the following steps:

Pepper seeds with similar sizes were selected, were soaked by 5% sodium hypochlorite (NaClO) for sterilization for 10 min, and were cleaned with deionized water for 5 min, the sterilized and cleaned seeds were soaked for 6 h in deionized water, and were then placed onto wet filter paper for germination for 6 days without light. Then, the seeds were transferred into each flowerpot containing 2 kg of soil, the soil for experiments was taken from farmland soil in Wuxi, China (longitude 120.28° E, latitude 31.48° N). The total nitrogen, total carbon and pH value of the soil were respectively 11.76 g kg⁻¹, 149.46 g kg⁻¹, and 7.36. The flowerpot was placed in a climate chamber of Jiangnan University (temperature: 25° C., relative humidity: 60±5%).

In the flowering period of pepper, 10 mg/L of a Fe—P NMs aqueous solution was sprayed onto the front leaf surfaces and back leaf surfaces of pepper plants every day, the spray is performed for four times, and the spray application time was 9 a.m. to 11 a.m. A sunny and mild weather was preferred, the uniform spray application onto the front leaf surfaces and back leaf surfaces was possibly ensured, and no rain within 4 h after the foliage spray was possibly ensured.

In the test period of normal growth of pepper, other management measures were not adopted, and other possible interference factors were possibly eliminated. Sampling was performed after most pepper plants bore fruits.

### Example 3

The concentrations of the Fe—P NMs aqueous solutions in Example 2 were respectively adjusted to 1 mg/L and 50 mg/L, and the others kept constant with those in Example 2.

The spray application period in Example 2 was adjusted into the seedling period and the fruiting period, the concentrations of the Fe—P NMs aqueous solutions were respectively 1 mg/L, 10 mg/L, and 50 mg/L, and others kept constant with those in Example 2.

### Comparative Example 1 (CK)

The Fe—P NMs in Example 2 was omitted, and was replaced with pure water, and the others kept constant with those in Example 2.

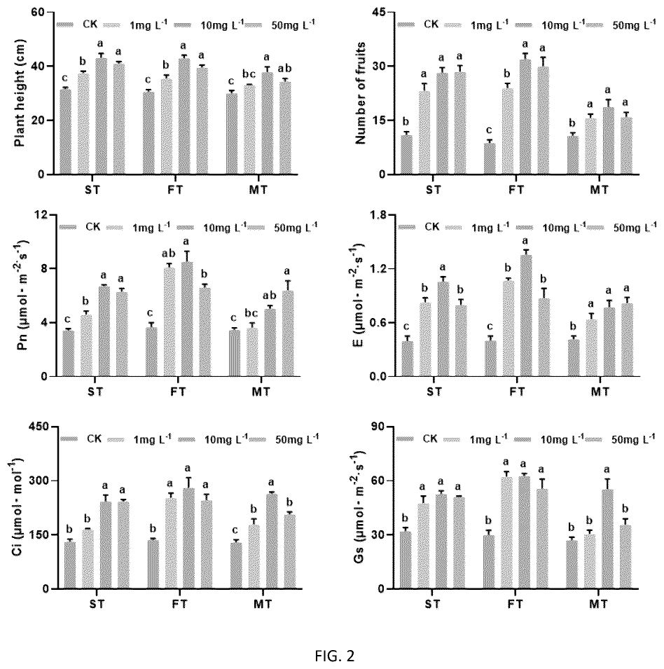

FIG. 2 shows pepper growth indexes under treatment with Fe—P NMs of different concentrations in different periods, the pepper growth indexes include the height of pepper plants, the number of pepper fruits, the net photosynthetic rate (Pn), the transpiration rate (E), the intercellular carbon dioxide concentration (Ci), and the stomatal conductance (Gs).

From FIG. 2, it could be seen that after the Fe—P NMs of different concentrations were sprayed and applied onto the leaves of the pepper plants, the growth and the yield of the pepper were increased to different degrees. The improvement effect of the 10 mg/L Fe—P NMs in the flowering period on the growth and the yield of the pepper was the optimum, and the specific effect was as follows: compared with the treatment with CK, the treatment with the 10 mg/L Fe—P NMs aqueous solution in the flowering period was the optimum, the plant height was increased by 40.4%, the number of fruits was increased by 2.7 times, and additionally, the net photosynthetic rate, the transpiration rate, the intercellular carbon dioxide concentration, and the stomatal conductance were respectively increased by 1.3 times, 2.4 times, 1.1 times, and 1.1 times.

### Comparative Example 2 Same Number of Ions

3.14 mg L⁻¹ of P₂O₅ and 22.3 mg L⁻¹ of a Fe-EDTA solution, and 10 mg L⁻¹ of a solution with the same Fe and P amount, labeled as Ion.

Then, the solution was applied onto the leaves of the pepper plants according to the application method in Example 2.

Example 2 and Comparative examples 1 and 2 were subjected to property test, and the test results were as follows:

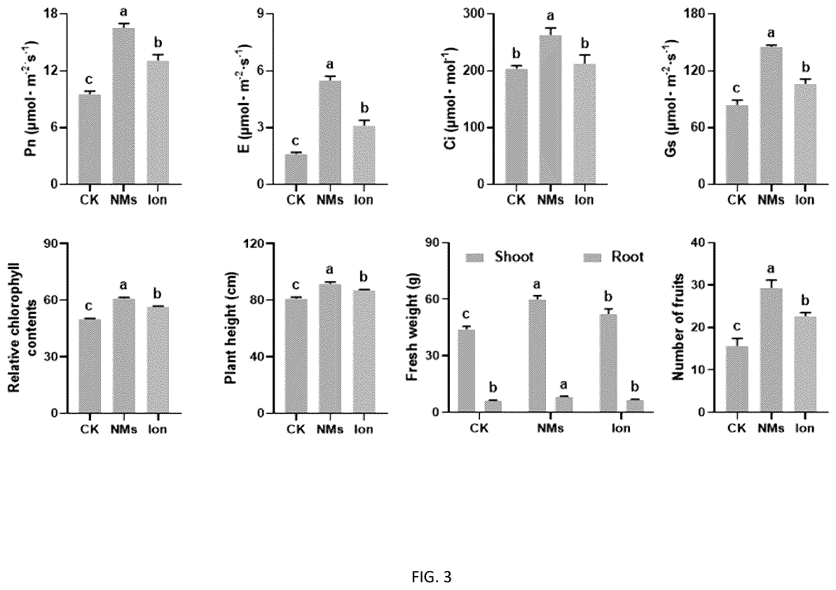

FIG. 3 shows property comparison of Example 2 and Comparative examples 1 to 2. From FIG. 3, it could be seen that compared with CK (Comparative example 1) and the same number of ions (Comparative example 2), the photosynthetic parameter, the chlorophyll content, the plant height, the fresh weight and the number of fruits under the treatment with 10 mg/L Fe—P NMs were the optimum, and the promoting effect was the most significant.

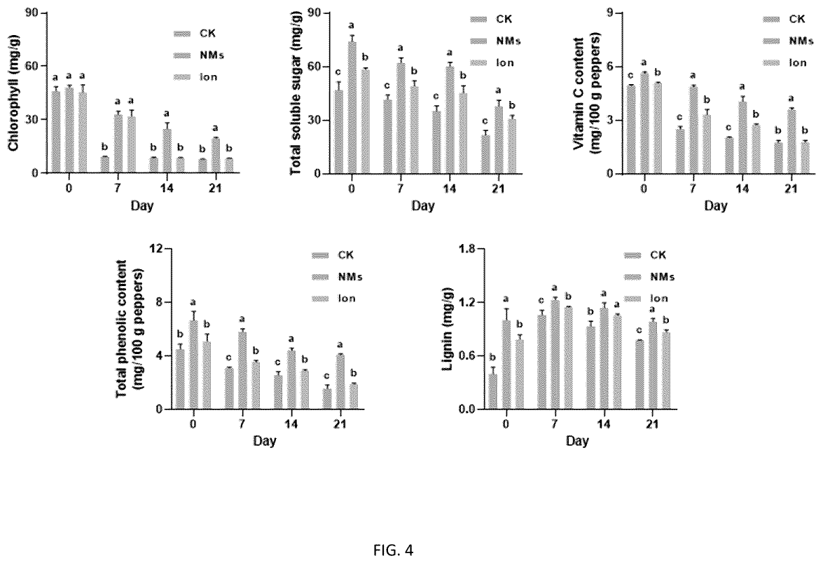

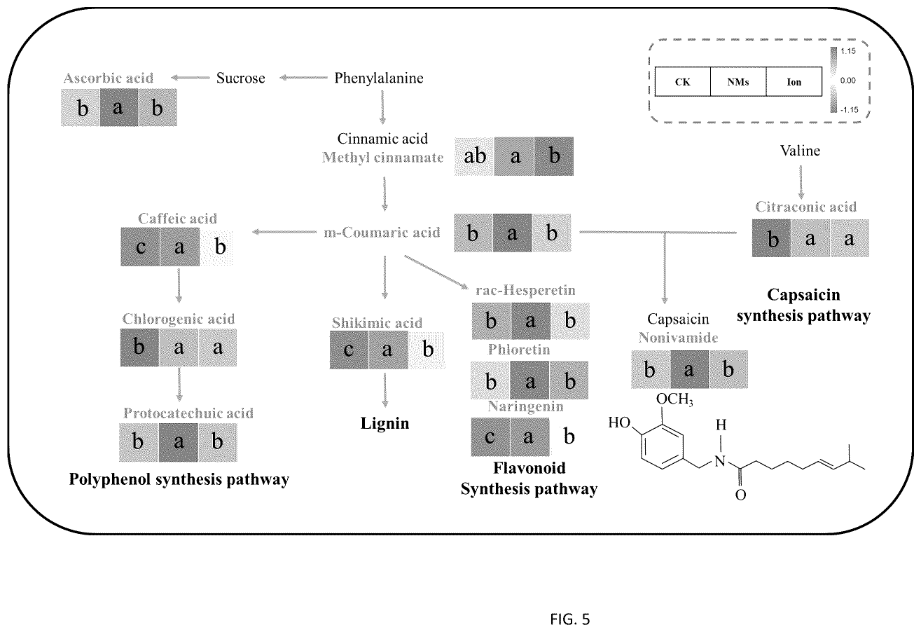

In the storage process of the pepper (temperature: 25°, and humidity: 70±5%), the content change of the chlorophyll, the soluble sugar, the vitamin C, the total phenols, and the lignin might reflect the aging degree and quality change of the pepper fruits. From FIG. 4, it could be seen that in the whole storage period, the chlorophyll content of the pepper fruits was in a decrease trend, the chlorophyll content of the pepper fruits under the NMs treatment was significantly higher than that of the CK group and the Ion group, and it showed that the NMs treatment has the inhibition effect on the chlorophyll content decrease in the pepper fruit storage period. Compared with the CK group, the NMs treatment and the Ion treatment respectively increased the soluble sugar content of the pepper fruits by 57.5% and 24.5%. After the storage for 21 days, the soluble sugar contents of the CK treatment, NMs treatment and Ion treatment were respectively decreased by 53.7%, 48.8% and 47.2% compared to the values just after the picking. In this case, the soluble sugar content in the fruits after NMs treatment and Ion treatment was respectively 74.4% and 41.9% higher than that of CK group. Identically, through the NMs treatment and the Ion treatment on the pepper fruits just picked, the vitamin C content of the pepper fruits was respectively increased by 13.8% and 2.8% compared to the CK group. After the storage for 21 days, the vitamin C content of the fruits after CK, NMs and Ion treatment was respectively reduced by 64.4%, 35.9% and 65.2%. In this case, the vitamin C content of the NMs treatment was 105.0% higher than that of the CK group, and the vitamin content of the Ion treatment was not significantly different from that of the CK treatment. The total phenolic content also had similar change trend. After the storage for 21 days, the total phenolic contents of the pepper fruits after NMs and Ion treatment were respectively 161.7% and 22.0% higher than that of the CK group. At each time point of the storage period, the lignin content of the pepper fruits after the NMs treatment was higher than that of the CK treatment and Ion treatment, and after the storage for 21 days, the lignin content of the pepper fruits after NMs treatment was respectively 27.0% and 13.8% higher than that of the CK treatment and Ion treatment.

FIG. 5 shows the change of a metabolic profiling of pepper fruits after the treatment with Fe—P NMs. In the synthesis route of total phenolic compounds, under the NMs treatment, the contents of caffeic acid, chlorogenic acid, and protocatechuic acid were respectively increased by 34.3%, 97.2% and 65.6% compared to CK treatment. The phenolic compounds around the plant infected tissues might effectively inhibit the propagation of pathogenic bacteria, so that the injury on plants caused by the pathogenic bacteria was reduced. In addition, compared to CK, the NMs treatment realized the increase of shikimic acid of precursor substances biologically synthesized by lignin in the pepper fruits by 36.8%, and it directly led to the lignin level increase in the pepper fruits. The lignin was a phenylpropane polymer, and was a main structural ingredient of secondary vascular tissues and fiber of higher plants. The lignin acted with cellulose to jointly enhance cell walls, and the resistance of fungi penetrating through the cell walls of plants was improved. After the foliage application of 10 mg L⁻¹ Fe—P NMs in the flowering period, the relative abundance of the flavonoid compounds in the pepper fruits was improved. The relative abundances of hesperetin, phloretin, and naringenin were respectively increased by 36.8%, 22.7% and 288.6% compared to the CK group. The flavonoid compounds, as special metabolites in horticultural plants, have important health promotion effects, and may clear free radicals and protect the plants from oxidative damage. After the foliage application of 10 mg L⁻¹ Fe—P NMs in the flowering period, the capsaicin content in the pepper fruits was increased by 65.6% compared to the CK group. The capsaicin is a substance obtained through vanillylamine condensation, originating from a phenylpropane pathway and a branched-chain fatty acid pathway. The capsaicin is a special substance in the pepper fruits, and is favorable for protecting the fruits from biotic and abiotic stress. Based on the above, through the foliage application of 10 mg L⁻¹ Fe—P NMs in the flowering period, the phenylpropane pathway and a branched-chain fatty acid pathway of the pepper fruits were influenced, finally, the biosynthesis of the capsaicin compounds was increased, the relative abundances of the phenolic compounds, the flavonoid compounds and the capsaicin compounds with the antibacterial and antioxidant effects were improved, and the maintenance of the appearance, the nutritional quality and the flavor of the pepper fruits in the storage period was facilitated, so that the storage period of the pepper was prolonged.

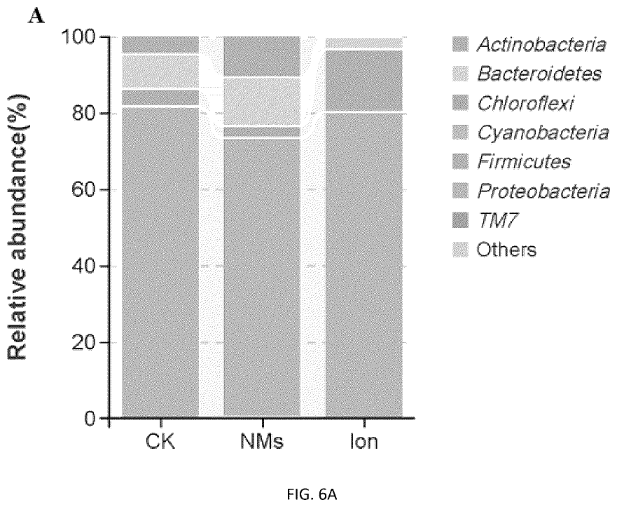

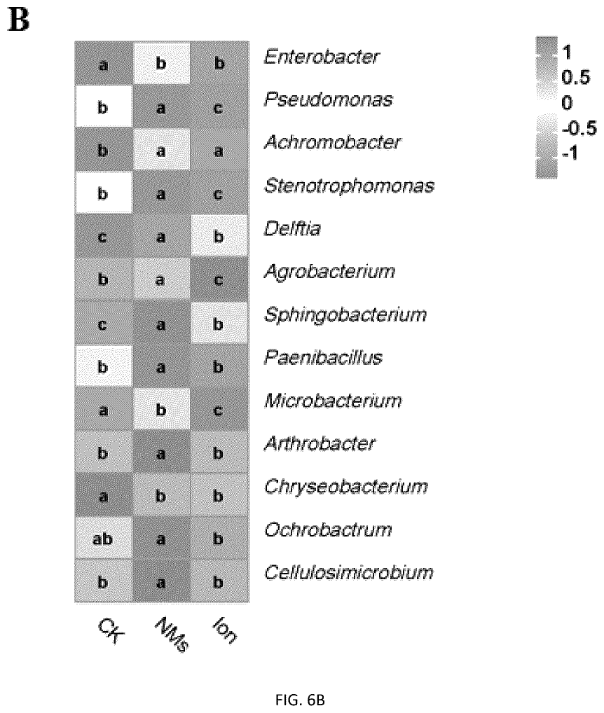

Dominant phyla of bacterial communities in pepper fruits of all treatment groups were Proteobacteria (78.2%), Bacteroidetes (8.3%), Firmicutes (8.1%), and Actinobacteria (5.3%), and the abundances of other phyla, for example, Chloroflexi, Cyanobacteria, and TM7 were relatively low. At the phylum level, the abundances of the Proteobacteria, the Bacteroidetes, the Firmicutes, and the Actinobacteria in the pepper fruits in the storage period were relatively high, the same results also occurred in fruits of apples, peaches, grapes, etc. The abundance of the Proteobacteria was the highest in all treatment groups, was the dominant phylum in the mature fruits, and were favorable for various metabolic activities, such as degradation of carbohydrate, amino acid and lipids. Compared to the CK group, the NMs treatment group respectively improved the abundances of the Bacteroidetes and the Actinobacteria in the fruits by 43.4% and 123.3%, they might synthesize biologically active compounds with antimicrobial activities, and decreased the abundance of the Proteobacteria unfavorable for the fruit preservation by 10.4%. At the genus level, compared with the CK group, the NMs group respectively decreased the abundances of Enterobacter and Chryseobacterium in the pepper fruits by 41.5% and 99.7%. Studies showed that the Enterobacter and the Chryseobacterium might cause fruit decay. Under the NMs treatment, the abundances of Pseudomonas and Arthrobacter were respectively improved by 80.3% and 96.6% compared to the CK treatment, and the Pseudomonas and the Arthrobacter might be used as biocontrol agents for effectively preventing and controlling the post-harvest fruit diseases. It is currently widely considered that the antagonistic mechanism of the biocontrol agents on the pathogenic bacteria includes competing for nutrition and space and generating secondary chemical resistance metabolites. In addition, compared to the CK group, the NMs treatment group improved the abundances of Sphingobacterium and Paenibacillus in the fruits by 97.3% and 150.0%. The Sphingobacterium and the Paenibacillus might generate amylase, protease, and chitinase, and these enzymes attacked cell walls of fungi, and the fruit injury was relieved by fungus pyrolysis through chitin degradation. In a word, through the foliage application of 10 mg L⁻¹ Fe—P NMs in the flowering period, the abundances of bacterial genera (Enterobacter and Chryseobacterium) prone to decay in the pepper fruits in the storage period might be reduced, the abundances of beneficial bacterial genera (Pseudomonas, Arthrobacter, Sphingobacterium, and Paenibacillus) might be improved, and a micro-ecological environment (FIGS. 6A and 6B) favorable for the preservation of the pepper fruits was finally formed.

Although exemplary examples of the disclosure have been disclosed above, they are not intended to limit the disclosure. Anyone familiar with the technique can make various modifications and improvements within the spirit and scope of the disclosure. Therefore, the protection scope of the disclosure shall be determined by the definition in the claims.

What is claimed is:

1. A method for promoting preservation of pepper using an iron-phosphorus nanomaterial, comprising: making the iron-phosphorus nanomaterial into an iron-phosphorus nanomaterial solution, and applying the iron-phosphorus nanomaterial solution onto leaves of pepper plants through foliage spray.

2. The method according to claim 1, wherein
a method for preparing the iron-phosphorus nanomaterial comprises the following steps:
respectively dissolving polyvinylpyrrolidone, a water-soluble trivalent iron salt, and a water-soluble phosphorus source in water to obtain a polyvinylpyrrolidone solution, an iron salt solution, and a phosphorus source solution; and
mixing the polyvinylpyrrolidone solution with the phosphorus source solution, then, dropwise adding the iron salt solution while stirring, after dropwise addition, continuously stirring for reaction for a period of time, then performing centrifugation, collecting a precipitate, transferring the precipitate into a reaction vessel, adding water for taking a hydrothermal reaction, after the reaction is completed, performing centrifugation, collecting solids, and performing washing and drying to obtain the iron-phosphorus nanomaterial, wherein
a mass ratio of the water-soluble trivalent iron salt to the water-soluble phosphorus source is 1:(1.5-2.5).

3. The method according to claim 1, wherein the iron-phosphorus nanomaterial has a width of 60 nm to 100 nm, a hydraulic diameter of 196.97±55.43 nm, and a Zeta potential of 16.33±0.80 mV.

4. The method according to claim 1, wherein a concentration of the iron-phosphorus nanomaterial solution is 1 mg/L to 50 mg/L.

5. The method according to claim 1, wherein a concentration of the iron-phosphorus nanomaterial solution is 10 mg/L.

6. The method according to claim 1, wherein an application amount of the iron-phosphorus nanomaterial solution is 5 mL/plant, an application frequency is once per day, totaling 4 times, and spraying time is between 9 a.m. to 11 a.m.

7. The method according to claim 1, wherein an application period is a seedling period, a flowering period, or a fruiting period.

8. The method according to claim 1, wherein a method for preparing the iron-phosphorus nanomaterial comprises the following steps:
respectively dissolving polyvinylpyrrolidone, a water-soluble trivalent iron salt, and a water-soluble phosphorus source in water to obtain a polyvinylpyrrolidone solution, an iron salt solution, and a phosphorus source solution; and
mixing the polyvinylpyrrolidone solution with the phosphorus source solution, then, dropwise adding the iron salt solution while stirring, after dropwise addition, continuously stirring for reaction for a period of time, then performing centrifugation, collecting a precipitate, transferring the precipitate into a reaction vessel, adding water for taking a hydrothermal reaction, after the reaction is completed, performing centrifugation, collecting solids, and performing washing and drying to obtain the iron-phosphorus nanomaterial.

9. The method according to claim 8, wherein a mass ratio of the polyvinylpyrrolidone to the water-soluble trivalent iron salt is 1:(4-5).

10. The method according to claim 8, wherein a mass ratio of the water-soluble trivalent iron salt to the water-soluble phosphorus source is 1:(1.5-2.5).

11. The method according to claim 8, wherein a speed of dropwise adding the iron salt solution is one drop per 6 second.

12. The method according to claim 8, wherein the hydrothermal reaction is conducted at a temperature of 150° C. to 200° C. for a duration of 3 hours to 8 hours.

13. A method for improving contents of lignin, total phenols, flavonoids, and capsaicin in fruits, and activities of antioxidant enzymes, comprising using an iron-phosphorus nanomaterial for improving the contents of lignin, total phenols, flavonoids, and capsaicin in pepper fruits, and the activities of antioxidant enzymes through promoting phenylpropane metabolism and capsaicin metabolism pathway changes of fruits;
wherein a method for preparing the iron-phosphorus nanomaterial comprises the following steps:
respectively dissolving polyvinylpyrrolidone, a water-soluble trivalent iron salt, and a water-soluble phosphorus source in water to obtain a polyvinylpyrrolidone solution, an iron salt solution, and a phosphorus source solution; and
mixing the polyvinylpyrrolidone solution with the phosphorus source solution, then, dropwise adding the iron salt solution while stirring, after dropwise addition, continuously stirring for reaction for a period of time, then performing centrifugation, collecting a precipitate, transferring the precipitate into a reaction vessel for heating reaction, after the reaction is completed, performing centrifugation, collecting solids, and performing washing and drying to obtain the iron-phosphorus nanomaterial.

14. The method according to claim 13, wherein the iron-phosphorus nanomaterial is made into an iron-phosphorus nanomaterial solution to be applied onto leaves of pepper plants through foliage spray.

15. The method according to claim 13, wherein a concentration of the iron-phosphorus nanomaterial solution is 1 mg/L to 50 mg/L, an application amount of the iron-phosphorus nanomaterial solution is 5 mL/plant, an application frequency is once per day, totaling 4 times, spraying time is between 9 a.m. to 11 a.m., and an application period is a flowering period.

16. A method for preservation by adjusting microorganism distribution in pepper fruits, comprising using an iron-phosphorus nanomaterial and increasing relative abundances of microorganisms related to decay in pepper fruits are decreased, and relative abundances of beneficial microorganisms;
wherein a method for preparing the iron-phosphorus nanomaterial comprises the following steps:
respectively dissolving polyvinylpyrrolidone, a water-soluble trivalent iron salt, and a water-soluble phosphorus source in water to obtain a polyvinylpyrrolidone solution, an iron salt solution, and a phosphorus source solution; and
mixing the polyvinylpyrrolidone solution with the phosphorus source solution, then, dropwise adding the iron salt solution while stirring, after dropwise addition, continuously stirring for reaction for a period of time, then performing centrifugation, collecting a precipitate, transferring the precipitate into a reaction vessel for heating reaction, after the reaction is completed, performing centrifugation, collecting solids, and performing washing and drying to obtain the iron-phosphorus nanomaterial.

17. The method according to claim 16, wherein the microorganisms related to decay in pepper fruits comprise Enterobacter and Chryseobacterium, and the beneficial microorganisms comprise Pseudomonas, Arthrobacter, Sphingobacterium and Paenibacillus.

18. The method according to claim 16, wherein the iron-phosphorus nanomaterial is made into an iron-phosphorus nanomaterial solution to be applied onto leaves of pepper plants through foliage spray.

19. The method according to claim 16, wherein a concentration of the iron-phosphorus nanomaterial solution is 1 mg/L to 50 mg/L, an application amount of the iron-phosphorus nanomaterial solution is 5 mL/plant, an application frequency is once per day, totaling 4 times, spraying time is between 9 a.m. to 11 a.m., and an application period is a flowering period.
